SEQUENCE LISTING (34 sequences)

SEQ ID NO: 1 - DNA, 654 bp, MORTIERELLA ALPINA
atggccatcaaggaataccagcgcgagttcattgagtttgccatcaagaacgaggtcttgaagttcggagagttcaccctcaagtccggccgtatctcgccctacttcttgaacgcgggccttttcaacactggcgcctcgctctccaagatcggaaagttctacgccgctgccgtcagcgattcgggcattgagcacgacatcatctttggccccgcctacaagggtgtccctctggcctgcaccaccgtcattgccttggccgaggccccctacaacaaggacacgccctactgcttcaaccgcaaggagaagaaggaccatggtgagggcggcacgattgttggatcagcactgaagggcaaggtcctggtcattgacgatgttatcaccgccggcaccgccatccgcgagtctgtccagatcattgaggactgcaaggcccaattggctggtgttttggtcgcggtggatcgtcaggagactggcaagaacggcgacatgtctgctatccaggaggtcgagagagactttggtgtccctgtcaaggccattgtgaccatgacccacatcatgcagtacatggaggagaagggaacctatggcgagcacttgacccagatgcgcgcttaccgggagaagtacggtgtttag

SEQ ID NO: 2 - DNA, 1752 bp, MORTIERELLA ALPINA
atgactgtcagcgaaaacaccactcataatatccagcagatctcaaagtcgtcgtatctcaaccagagtactgcgacacccactgagctccgctccgctctccacctccaaggtctgactcctgccaaggtcgagtcttttgagcttcagaagaagcgcgctctggcccagctcagaagcaagtcgtcagacattgaaaaatacgtctttctggcctggctcaggaacacaaacgtccgcctcttctatggtttggtgggcgatcagcttgaggaaaccctgcccctgatctatacacccacagtcggcaccgcatgccaaaactactctagcatctatcctttccttgcccctcctggtcaacctgatggtctcttcctctccatcaacgatctccccaacttgactcagatcatccagaactacaagcctttccctcaagatcctagcctcacgccacagatcgccgtcattaccgatggctcccgcattctgggcctgggagaccttggtgttggcggcatgggtatccctgttggaaagctacagctctatgttgctggagcaggaatcgatcccaggcgcacactccccatcacccttgacctgggcaccaataatgaggacaagttgaaggatgagttttatttgggcttgcgcgagaagcgtgctggtgatgacaagttcttccccttcgtcgacgctgttatcgaggccctcacctcgttgtaccccaagctcctgatccagttcgaggacttctcctcagaacacgccttccagttgctcgacaagtaccaacccaaaaagttctgcttcaacgatgacatccaaggaactggtgccgtcatcctggccggatttatgaacgcgatcaaccttgccggcatccctgccaaggaccacaagattctctttttcggtgcaggttcagccgctgtcggtgtggccaagcagcttgctgcctactttgtcaatgaccacggcatgactgaggacgaggctcgcgagatggtctggttggtcgactccaaaggtttggtgacgctggaccgtggtgacaagctggctgaacacaagctatactttgctcgcaaggacaatgccggaagccagtaccctagcattgcatctgttatcgagcacgttcgtcctacagccttgttcggcctctcatcacaatctggtgccttctctgaagacgtgctcaagtccatggccaccctgaaccagcgccctattgtcttccccttgtcgaaccccgcctaccaagccgagtgctcattcgagcaggctatgatccacaccaagggcaaggtgatctttgcctctggcaccgcgttccccaagtacaccgaccccaacaccggcctcatcagcgcacctggccagggcaacaacatgtacatcttccctggtctcggattgggaggtattttggcccagcctgagaccatcagcgaccgcctcatctattcggctgctcgcgcctgcgccaatgcattgactgtcgaggagcgctcccaaggcctgctctaccctgtcttgcctcgcatccgtcaggtgtccgctgaggttgcagccgctgttgtgactgaggctgttgccgaagggactgcaacgaacgaggaggccatcaagctggtcaagtctcacgacaaggcggcgcttctgaactttgtcaacagcaatatgtggaccaccgatggggtccagcatgaaatcagcaccacctttgcccctcacctctaa

SEQ ID NO: 3 - DNA, 1857 bp, MORTIERELLA ALPINA
atgttgaggaatcctgctctcaagacggtccatgctgccacgcaacgctacgtcgctagccagacacgacactacagcagacgtgctctgaccaccctcgctgccttcccagacctgcgcatggaaacgatggtccagacgacgaccaaacacggtatggatgtcattcacgaccctctcctctccaaaggcaccgccttctcgctctcagaacgtgaacgtctcggcatccgcggcctggtgcccccaaggacccaggagatggacaagcagctccagcgcatcatccgcaacttgagagagtgccggacacctctcgaaaagttcattttcatgaccgccttgcaggaccgtaacgagaccctctactaccgcctactgatcgacaacttggaggagctttctcagatcatctacacacccactgtgggtttggcttgtcagcgcttccactccatttatcgtcgctccaggggcatgtatttcagcacgctggaccgtggtcaaatggcggctatggttcacaactggcctcacgatcaggtcgatgtcattgttgttacagatggatctcgcgtcttgggcctgggtgatttgggagcaaacggcatgcagatccccatcggaaagctttcattgtatgtcgctggaggaggaattcgtccaacctccatcttgcccgtggttctagatgtcggcactaataacgaggagctgctgaacgatcctttgtaccttggaatggggcaccgtcgtttggaaggcgaggaatactattcgttcgtagatgaatgggttgcagctattcagaacagatatcccaatgctctgattcagttcgaggactttaaatacccacatgcatacaacctcctccacaagtatcaagacaagatctgctgtttcaacgatgacattcaatcgacctccgctgtcgccttggctggtgtcttggcagctctgaaggctcgcagtttgcccattgataacctcaacgaggagcgtatcatttgcgtcggcgccggaagcgctggtgttggtgtctgcgagggaatcattgattgcatggtagagcagggcaaggtcaagtcgagagaggaggcctataagcgcatctggatgctcgatcagcatggtcttattggcaaccccaacctcggcacctcagacggtacaaaacctcctaccattggtctaggagatcgcgccaacttggatgagcgtcagctgtgctatctcaagaaggatatgcatgaccgtatgagccttgaggaggttgtgaaagtcatcaagcccacggttattcttggtctcagtggagctcctggtgtcttcacagagggggctatccgtgctatggccgatggtgtcgagaagcccgtggtattccctctttctaaccctacccatcaatccgaatgcactgccgagcaggccttcaagtggacggacggccgcgccatctttgccagcggaagccccttcaaggatgtcgagtacaagggcaaaatctgcaaagtgaatcagattaacaacgccatgtcgttccccggtctcggtctcggtgttacagtgtcgcggtcctcgcgtgtgacaggaaagatgttcatggagactgccatcactatcgccaacatggcatcagaggagcagcttaatagcgggatccttttccctggagttgctgcactgcgtgaggtctcgaaggaggttgcggcaagggtgtgcgaagttgcgtacgaccaaggcttggcccgtgctcagctgccccagggggttagtttgaagggtcttatcgaggagtcgatgtggaagcctgcgtacgtgcctacgattcgcctggaatcgcacccctga

SEQ ID NO: 4 - DNA, 140 bp, MORTIERELLA ALPINA
tcccaagcgaatttgtcatctcgactggtgcaaactgcgcaaacggctgactcattgcccatgcttttcttctccacgcctatcgtctgttactgcatctctgtcgtgttgaatgcgtaactgatggacttccgtatgct

SEQ ID NO: 5 - DNA, 30 bp, Artificial Sequence
  The oligo sequence is synthesized as primer for PCR analysis.
acatcatgaccatcaaggaataccagcgcg

SEQ ID NO: 6 - DNA, 28 bp, Artificial Sequence
  The oligo sequence is synthesized as primer for PCR analysis.
tcgggatccctaaacaccgtacttctcc

SEQ ID NO: 7 - DNA, 29 bp, Artificial Sequence
  The oligo sequence is synthesized as primer for PCR analysis.
catgcgtcatgactgtcagcgaaaacacc

SEQ ID NO: 8 - DNA, 31 bp, Artificial Sequence
  The oligo sequence is synthesized as primer for PCR analysis.
tacgcggatccttagaggtgaggggcaaagg

SEQ ID NO: 9 - DNA, 33 bp, Artificial Sequence
  The oligo sequence is synthesized as primer for PCR analysis.
atcggggtaccatgttgaggaatcctgctctca

SEQ ID NO: 10 - DNA, 31 bp, Artificial Sequence
  The oligo sequence is synthesized as primer for PCR analysis.
taattcccccgggtcaggggtgcgattccag

SEQ ID NO: 11 - DNA, 54 bp, Artificial Sequence
  The oligo sequence is synthesized as primer for PCR analysis.
gcatgccatggagaagcttggtaccgctagctcccaagcgaatttgtcatctcg

SEQ ID NO: 12 - DNA, 9666 bp, Artificial Sequence
  Plasmid pBIG2RHPH2 sequence
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattatcgatgaattcgacgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacccaagcatccaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctccagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggaaccagttaacgtctagaggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggcgtcgagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagactcgacgcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 13 - DNA, 654 bp, MORTIERELLA ALPINA; identical to SEQ ID NO: 1

SEQ ID NO: 14 - DNA, 654 bp, MORTIERELLA ALPINA; identical to SEQ ID NO: 1

SEQ ID NO: 15 - DNA, 1393 bp, MORTIERELLA ALPINA
cgacgctgacattacacatttatccgttcgccgatatagacttaaatgggacgaggagaacttgatcatcacagaggctcagaaggattcgacaatgaaaattgatgagcccaagacgccctatgttcactacgaccacgagctggacaaagtgatcgatatgaatggtaactgaaaacatggacatccagagatcttgagcacccacgacgagatacacagaaagtcatcaggcatactgacacgtccttcacgtcacgcatttctgaattttttaatttgtctaggggaaaccttctcgttagacgggggcaagacaaagcatgcttctctagcccatggtcagcctgtaccatcgcacatggatgaaccaatcggcgaggaagacgacgagagcgaggacgaagacgaggatgaagatggaccagacgagtcgggggattcagacgagggcgaggatgaagacgcaaaagaagagtcgcctcactaaaaaagtaagttttctctatctcattgcttctttggttgctcggataatgcttagctgttgttggtaaactcccagtagccaacgctcatcatttgcaatttttattttcgcatatatcagttgaccacgacaagtttgccaagatgcgtgcggaacactacaagatgaaggaggcgcttcaattggggcatgagctggcagaggaagagctgagtgcgctggacagtcctgatccaaacgatatgccagtgccgccattaccgtcgtttgctcaacagtcgaacgcggctaggctgtcacgggaggctggatcgaacaagctgaaggaggaccttgaaaacatggagctttagaggtttggagttggctttgaccatggctatggctacgtattctgaacgacataaaggacgctcatttttcgctgcaggacattttttgagttgcagcacagaggggcaaggcggtgctctggactgctttatcgggctgctacgcgtgcgatttgtttacgttttttccggtttgttggccagcagtatttgtaggccctgcagctgggggtgggttgatcctctttctcttctcttctcttttctctttttccctcttctgatgtgtctcccaccccacaaccttctcctctgcccccagccgcatcggtcccaccgccgcaacccatcagcacaccatggccatcaaggaataccagcgcgagttcattgagtttgccatcaagaacgaggtcttgaagttcggagagttcaccctcaagtccggccgtatctcgccctacttcttgaacgcgggccttttcaacactggcgcctcgctctccaagatcggaaagttctacgccgctgccgtcagcgattcgggcattgagcacgacatcatctttgg

SEQ ID NO: 16 - DNA, 1362 bp, MORTIERELLA ALPINA
ccctctggcctgcaccaccgtcattgccttggccgaggccccctacaacaaggacacgccctactgcttcaaccgcaaggagaagaaggaccatggtgagggcggcacgattgttggatcagcactgaagggcaaggtcctggtcattgacgatgttatcaccgccggcaccgccatccgcgagtctgtccagatcattgaggactgcaaggcccaattggctggtgttttggtcgcggtggatcgtcaggagactggcaagaacggcgacatgtctgctatccaggaggtcgagagagactttggtgtccctgtcaaggccattgtgaccatgacccacatcatgcagtacatggaggagaagggaacctatggcgagcacttgacccagatgcgcgcttaccgggagaagtacggtgtttagagcaagcgaactctggatgggatgaagctcggtttcaatgcggcgagcgagggctctgttggatttttctcgtaatgcggggagacggacgcccggggaacgatgtgctcctgatcagtggttttcgagtgttctcgggacagcccgtcttgggaaaccaccgaacgatggctattaataataaatacccatacaacaacttttcctcagtgtggtagttggggtgtgatatcgccgtgcatgtccaaggcttcagctgcgcctggcgacgagatggaaggtcgtgggaaagaggcgtcgaaactgagctgtcaagaagaaagtaaaaaaaccgtcgtaaaatagagctgtgtcgtcaaatggcgtgtatggggtattcggcgcgacaggctatttgattccgatggggctccagacaaggcgccaggagctcatccaagtcgatcgcccgctgtacgacgcctctgtagtatggggacatgattctgctggtggatgtttctgcagccaccagaaaattgaagctcagcctgtaaaaaaaaatattacattgtggcgagcgagtcacttctctgttctccttttcatttccacccaccctcattccacatccattcaccaccgctcattcgcttcacaatggcagagactcttactcaccctcttgtccaggacggctggttcaaggagaccggcaccctctggcccggccaggccatgactctcgaggtcaaggagattctgcacgttgaaaagtcgctcttccaggacgtgctcgtcttccagtccacctcctacggcaacgtcctcgtcctcgacggcgtcatccaggccaccgagcgcgatgagttctcgtaagtgcgctagtgtgctagtgtgtgctcgagctcctcacctggagtcctctacctgatcattccatatctgtgattcgcatg

SEQ ID NO: 17 - DNA, 45 bp, Artificial Sequence
  The oligo sequence is synthesized as primer for PCR analysis.
gagacgaattcgcccgtacggccgactagttttagttgatgtgag

SEQ ID NO: 18 - DNA, 45 bp, Artificial Sequence
  The oligo sequence is synthesized as primer for PCR analysis.
gttcctcgtctagacctctaaacaagtgtacctgtgcattctggg

SEQ ID NO: 19 - DNA, 36 bp, Artificial Sequence
  The oligo sequence is synthesized as primer for PCR analysis.
gaccggaattccgacgctgacattacacatttatcc

SEQ ID NO: 20 - DNA, 46 bp, Artificial Sequence
  The oligo sequence is synthesized as primer for PCR analysis.
gacggtggtgcaggccagagggccaaagatgatgtcgtgctcaatg

SEQ ID NO: 21 - DNA, 47 bp, Artificial Sequence
  The oligo sequence is synthesized as primer for PCR analysis.
ttgagcacgacatcatctttggccctctggcctgcaccaccgtcatt

SEQ ID NO: 22 - DNA, 31 bp, Artificial Sequence
  The oligo sequence is synthesized as primer for PCR analysis.
tgcggggtacccatgcgaatcacagatatgg

SEQ ID NO: 23 - DNA, 35 bp, Artificial Sequence
  The oligo sequence is synthesized as primer for PCR analysis.
tttcgctagcacgacgttgtaaaacgacggccagt

SEQ ID NO: 24 - DNA, 34 bp, Artificial Sequence
  The oligo sequence is synthesized as primer for PCR analysis.
aacaacaattggggctccaccgcggtggcggccg

SEQ ID NO: 25 - DNA, 23 bp, Artificial Sequence
  The oligo sequence is synthesized as primer for PCR analysis.
cacacacaaacctctctcccact

SEQ ID NO: 26 - DNA, 26 bp, Artificial Sequence
  The oligo sequence is synthesized as primer for PCR analysis.
caaatgaacgtatcttatcgagatcc

SEQ ID NO: 27 - DNA, 19 bp, Artificial Sequence
  The oligo sequence is synthesized as primer for PCR analysis.
gtgttcactcgcatcccgc

SEQ ID NO: 28 - DNA, 20 bp, Artificial Sequence
  The oligo sequence is synthesized as primer for PCR analysis.
aggcactctttgctgcttgg

SEQ ID NO: 29 - DNA, 19 bp, Artificial Sequence
  The oligo sequence is synthesized as primer for PCR analysis.
ggctgttgccgaagggact

SEQ ID NO: 30 - DNA, 21 bp, Artificial Sequence
  The oligo sequence is synthesized as primer for PCR analysis.
ggcaaaggtggtgctgatttc

SEQ ID NO: 31 - DNA, 21 bp, Artificial Sequence
  The oligo sequence is synthesized as primer for PCR analysis.
ccttgcaggaccgtaacgaga

SEQ ID NO: 32 - DNA, 21 bp, Artificial Sequence
  The oligo sequence is synthesized as primer for PCR analysis.
cctggagcgacgataaatgga

SEQ ID NO: 33 - DNA, 24 bp, Artificial Sequence
  The oligo sequence is synthesized as primer for PCR analysis.
cgtactaccgattgaatggcttag

SEQ ID NO: 34 - DNA, 25 bp, Artificial Sequence
  The oligo sequence is synthesized as primer for PCR analysis.
ccgtactaccgattgaatggcttag

(19) United States
(12) Patent Application Publication
(10) Pub. No.: US 2016/0289690 A1
(43) Pub. Date: Oct. 6, 2016

(54) MORTIERELLA ALPINA RECOMBINANT GENE EXPRESSION SYSTEM AND CONSTRUCTION METHOD AND USE THEREOF

(71) Applicant: JIANGNAN UNIVERSITY, Wuxi, Jiangsu (CN)

(72) Inventors: Yongquan CHEN, Wuxi, Jiangsu (CN); WEL CHEN, Wuxi, Jiangsu (CN); GUANGFEI HAO, Wuxi, Jiangsu (CN); HAIQIN CHEN, Wuxi, Jiangsu (CN); JIANXIN ZHAO, Wuxi, Jiangsu (CN); ZHENNAN GU, Wuxi, Jiangsu (CN); HAO ZHANG, Wuxi, Jiangsu (CN); DANHUI HAO, Wuxi, Jiangsu (CN); SHANSHAN ZHAO, Wuxi, Jiangsu (CN)

(21) Appl. No.: 15/032,639

(22) PCT Filed: Mar. 4, 2014
(86) PCT No.: PCT/CN2014/072839

§ 371 Date: Apr. 28, 2016

(30) Foreign Application Priority Data
Apr. 30, 2013 (CN) 201310524221.4

(51) Int. Cl. C12N 15/80; C12N 9/04

(52) U.S. Cl. CPC C12N 15/80; C12N 9/0006; C12Y 101/0104; C12Y 101/01038

(57) ABSTRACT

It relates to a Mortierella alpina recombinant gene expression system, to its construction method and application which is constructed by transformation of M. alpina ATCC 32222 uracil auxotroph strain through A. tumefaciens mediate transformation (ATMT) and is based on the existing uracil auxotrophic strain, through genetic engineering methods to obtain a final phenotype complementary strain to achieve the malic enzyme 1 and malic enzyme 2 overexpression strains.

This application is the U.S. national phase of International Application No. PCT/CN2014/072839 filed on 4 Mar. 2014 which designated the U.S. and claims priority to Chinese Application Nos. CN201310524221.4 filed on 30 Oct. 2013, the entire contents of each of which are hereby incorporated by reference.

### FIELD OF THE INVENTION

The present invention relates to a Mortierella alpina recombinant gene expression system, to its construction method and application. It is in the field of biotechnology engineering.

### BACKGROUND OF THE INVENTION

Mortierella alpina, an important oleaginous filamentous fungus, with the characteristics of high level of arachidonic acid (AA), safety and reasonable composition of polyunsaturated fatty acids (PUFAs), has been employed in the industrial production of AA. By far, the research on M. alpina is mainly focused on strain selection and optimization of fermentation conditions and so on. Due to the lack of an effective genetic manipulation system, M. alpina, such an extremely valuable filamentous fungi, cannot be subjected to the genetic transformation, which has constituted an insurmountable obstacle to the basic theoretical research of fatty acid synthesis pathway and production of strains in genetic engineering of M. alpina.

The progress of establishing gene manipulation system of filamentous fungi falls far behind of other species due to its characteristics of difficulty in being transformed. In particular, the fungus, which has the same features as those of Mortierella alpina, such as multi nucleus, no septum hypha, low yield of spore, as well as insensitivity to antibiotics, is one of the most difficult strains to be transformed. This may account for the absence of reports in gene modification of this industrial microorganism. Besides the characteristics and preferences of the host strain, the transformation methods also play an important role in the transformation rate.

Nowadays, there are mainly four kinds of transformation methods employed for filamentous fungi, including protoplast transformation (PT), electroporation transformation (ET), particle bombardment (PB) and Agrobacterium tumefecience mediated transformation (ATMT). PT and ET require a period of time consuming step-protoplast formation, what's more, the difficulty in cultivating and regenerating the protoplast results in low transformation rate. Compared with the above two methods, PB has quick and simple operation process. But the need for large amount of host cells and high expense impend its wide use. ATMT, originally used in plants transformation, was reported to conquer the ability to transform fungi twenty years ago. Up to now, it has been successfully established in over one hundred and twenty fungal strains. ATMT has several advantages: identifying various acceptor materials covering spores, sporangium and hypha and skipping protoplast formation step; high transformation rate; high capacity for heterologous DNA; randomly integrated into the host genome in one copy; increasing host homologous recombination rate. Therefore, ATMT offers an important manipulation method for the establishment of Mortierella alpina gene expression system.

Malic enzyme (EC 1.1.1.40) can catalyze malic to pyruvate, which is an source of NADPH, an important substance in microbes. In nineteen ninetieth, malic enzyme was inferred as a key factor in fatty acid synthesis in filamentous oleaginous fungi. According to Colin et al., the content of total fatty acid of Mucor circinelloides, a same member in Zygomycota as M. alpina, was dramatically affected by sesame, a chemical inhibitory for malic enzyme. During M. alpina fermentation, we observed the activities of a series of enzymes in NADPH producing process, among which, malic enzyme was intensively related to fatty acid synthesis. Without an effective gene manipulation system, this theory has not been testified in M. alpina yet.

The present patent uses the uracil auxotrophic strain M. alpina ATCC 32222 disclosed in the patent application 201310347934.8 as host strain, on the basis of which, through further gene recombination method, construct a genetic expression system which can highly express malic enzyme. The patent application No. 201310347934.8 is incorporated herein by reference in its entirety.

The technical solution disclosed in Chinese Patent Application 201310347934.8 involves a M. alpina uracil auxotroph that was constructed by inactivating the orotate phosphoribosyl transferase (OPRTase) coding gene ura5 in M. alpina ATCC 32222 genome.

About the M. alpina uracil auxotrophic strain, the inactivation of ura5 gene was achieved by deletion of the 18 bp (213 bp to 230 bp) of the 654 bp ura5 genome DNA.

Chinese Patent Application No. 201310347934.8 also disclosed a process for preparing the above M. alpina uracil auxotrophic strain, which involves inactivating the M. alpina ura5 gene through deletion of the 18 bp (213 bp to 230 bp) DNA sequence via homologous recombination. The homologous DNA arms are the 1393 bp (from −1180 bp to +212 bp) up-stream and the 1362 bp (from +23 lbp to +1592 bp) down-stream of the ura5 gene. The detailed steps of the said method are described as follows: obtaining a ura5 knockout DNA fragment and further constructing the knockout plasmid pBIG4KOura5; transformation of A. tumefaciens using pBIG4KOura5; transforming M. alpina with transformed A. tumefaciens harboring plasmid pBIG4KOura5; screening and identifying the transformed M. alpina to obtain M. alpina uracil auxotrophic strains.

The A. tumefaciens used in the said method is Agrobacterium tumefaciens C58C1.

The starting A. tumefaciens plasmid is pBIG2RHPH2.

The gene knockout plasmid is constructed as described below:

1) amplifying MCS DNA fragment by PCR with the plasmid pBluescript II SK+ as template;

2) digesting MCS gene fragment and plasmid pBIG2RHPH2 with EcoRI and XbaI and inserting the MCS gene fragment into EcoRI and XbaI sites of plasmid pBIG2RHPH2 through the ligation reaction to form the plasmid pBIG4;

3) PCR amplifying the up- and down-stream arms of ura5 gene and ligating them with each other by using fusion PCR to form knockout gene fragment;

4) digesting the KOura5 knockout gene fragment and pBIG4 with EcoRI and KpnI, and ligating them together to form plasmid pBIG4KOura5.

Preferably, the knockout gene fragment in step 3) is constructed as the following steps:

The primers are designed according to the sequence data of NCBI

P1:
(SEQ ID NO. 19)
GACCGGAATTCCGACGCTGACATTACACATTTATCC
P2:
(SEQ ID NO. 20)
TGACGGTGGTGCAGGCCAGAGGGCCAAAGATGATGTCGTGCTCAATG
P3:
(SEQ ID NO. 21)
TTGAGCACGACATCATCTTTGGCCCTCTGGCCTGCACCACCGTCATT
P4:
(SEQ ID NO. 22)
TGCGGGGTACCCATGCGAATCACAGATATGG

Subsequently, up- and down-stream DNA fragments are PCR amplified using the primer P1 and P2, and P3 and P4 respectively, with M. alpina ATCC 32222 genome DNA as template. Fusion PCR is performed using P1 and P4 with up- and down-stream DNA fragments as templates to amplify the KOura5 knockout DNA sequence.

More preferably, the primers below are designed according to the sequence of pBluescript II SK⁺:

up-stream of MCS:
(SEQ ID NO. 23)
TTTCGCTAGCACGACGTTGTAAAACGACGGCCAGT
down-stream of MCS:
(SEQ ID NO. 24)
AACAACAATTGGGGCTCCACCGCGGTGGCGGCCG

Then the MCS gene fragment of the plasmid pBluescript II SK⁺ in step 1) is amplified by PCR.

Preferably, the said ATMT gene knockout is to use A. tumefaciens to transform M. alpina, specified as: mixing equal volume of 100 μL of A. tumefaciens and M. alpina spores, and spreading on the cellophane membrane placed on the IM solid medium. After co-cultivation, select the uracil auxotrophic strains of M. alpina.

Preferably, the ATMT method is detailed below:

(1) separating the A. tumefaciens harboring pBIG4KOura5 (preserved at −80° C.) by stripping on the YEP solid plate (containing 100 μg/mL rifampicin and 100 μg/mL kanamycin) to obtain single clone by culturing at 30° C. for 48 h;

(2) transferring a single clone to 20 mL YEP medium (containing 100 μg/mL rifampicin and 100 μg/mL kanamycin) and culturing at 30° C. for 24-48 h with shaking at 200 rpm in the dark;

(3) collecting A. tumefaciens by centrifuging at 4000×g for 5 min, after removing the suspension, suspending the pellet by 5 mL of IM medium, followed by a centrifugation at 4000×g for 5 min, after removing the suspension, then adding 2 mL of IM medium to suspend the bacterium;

(4) adjusting the concentration of the bacterium suspension to OD₆₀₀=0.9, followed by a dark cultivation at 30° C. to OD₆₀₀=1.5;

(5) collecting the M. alpina spores and counting the number, then adjusting the spore concentration to 106/100 μL;

(6) mixing the equal volume of 100 μL of A. tumefaciens and spores and spreading on the cellophane membrane placed on the IM solid medium, then incubating at 23° C. for 48 to 96 h in the dark;

(7) transferring the cellophane membrane onto GY plate containing 100 μg/mL cefotaxime, 100 μg/mL spectinomycin and 0.05 g/L uracil, then incubating at 25° C. to 30° C. until spores appears.

The A. tumefaciens C58C1-pBIG4KOura5 generated according to Chinese Patent Application No. 201310347934.8 has been maintained in China General Microbiological Culture Collection Center (CGMCC) since Jun. 28, 2013. The address of CGMCC is the Institute of Microbiology, Chinese Academy of Sciences, No. 1, Beichen West Road, Chaoyang District, Beijing, China, Zip code 100101. The accession number is CGMCC No. 7730.

### SUMMARY OF THE INVENTION

The object of the present invention is to provide a M. alpina recombinant gene expression system. The said M. alpina recombinant gene expression system is constructed by transformation of M. alpina ATCC 32222 uracil auxotroph strain through A. tumefaciens mediate transformation (ATMT).

The selective marker ura5 gene, malE1 gene and malE2 gene are obtained from the genome DNA sequence of M. alpina ATCC 32222 (DDBJ/EMBL/GenBank accession ADAG00000000, first version ADAG01000000).

The present invention also provides a method for constructing a M. alpina recombinant gene expression system. As illustrated in FIG. 1, the said method comprises: obtaining the HPH expression cassette from PD4 by PCR method, and digesting the resultant HPH expression cassette with EcoRI and XbaI, inserting the digested HPH into the MCS site of pET28a (+) digested by EcoRI and XbaI, to form the plasmid pET28a-HPHs; obtaining the ura5 (orotate phosphoribosyl transferase; OPRTase) gene from M. alpina cDNA by PCR, then digesting the resultant ura5 with BspHI and BamHI, subsequently inserting the digested ura5 into the digested pET28a-HPHs with NcoI and BamHI to replace hpt gene to form pET28a-ura5s; obtaining the ura5 expressing cassette was by digesting with EcoRI and XbaI; taking the place of HPH expression cassette in the plasmid pBIG2RHPH2 with ura5s expression cassette to further form plasmid pBIG2-ura5s; Then transforming A. tumefaciens with the plasmid pBIG2-ura5s, finally transforming M. alpina uracil auxotroph with transformed A. tumefaciens containing pBIG2-ura5s, and screening and identifying the transformed M. alpina to obtain the positive transformants.

Further, the malic enzyme 1 overexpression plasmid is constructed based on pBIG2-ura5s. The malE1 gene is amplified from the cDNA of M. alpina. The malE1 gene is digested with BspHI and BamHI, and pET28a-HPHs is digested with NcoI and BamHI, respectively, then malE1 gene fragment is inserted into NcoI and BamHI sites of the plasmid pET28a-HPHs to form plasmid pET28a-malE1; the malE1 expressing cassette is obtained by digesting pET28a-malE1 with SpeI and XbaI. Then malE1 expressing cassette is inserted into pBIG2-ura5s digested with XbaI to form pBIG2-ura5s-malE1. Then A. tumefaciens is transformed with the plasmid pBIG2-ura5s. M. alpina uracil auxotroph is transformed with transformed A. tumefaciens C58C1 containing pBIG2-ura5s-malE1. And then the transformed M. alpina is screened and identified to obtain phenotype complementary strains MA-malE1-1, MA-malE1-2 and MA-malE1-3, thus construct homologous overexpression of malic enzyme 1 gene in M. alpina.

Even further, the M. alpina gene manipulation universal vector is constructed based on the plasmid pBIG2-ura5s and pET28a-HPHs. As illustrated in FIG. 2, the IT noncoding DNA sequence is PCR amplified from M. alpina genome. IT gene fragment and pET28a-HPHs is digested with NcoI and BamHI, respectively, then replace hpt gene in the pET28a-HPHs with IT fragment by ligation reaction, to form pET28a-ITs. The ITs expression cassette is obtained by digesting pET28a-ITs with SpeI and XbaI, then ligated into pBIG2-ura5s digested with XbaI to form the M. alpina gene manipulation universal vector pBIG2-ura5s-ITs.

Still further, the malE2 overexpressing vector is constructed based on the M. alpina gene manipulation universal vector pBIG2-ura5s-ITs. The malE2 gene and pBIG2-ura5s-ITs are double digested with KpnI and XmaI, respectively and then ligated with ligase to form malE2 expression plasmid pBIG2-ura5s-malE2. Then A. tumefaciens is transformed with the transformant plasmid pBIG2-ura5s-malE2. Finally M. alpina uracil auxotroph is transformed with transformed A. tumefaciens C58C1 pBIG2-ura5s-malE2 containing pBIG2-ura5s-malE2. The transformed M. alpina is selected and identified to obtain phenotype complementary strains MA-malE2-1, MA-malE2-2 and MA-malE2-3, thus construct homologous overexpression of malic enzyme 2 gene in M. alpina.

In particular, the present invention provides a M. alpina recombinant gene expression system, which is constructed by ATMT of Mortierella alpina ATCC 32222 uracil auxotrophic strain. Based on this system, the M. alpina malic enzyme 1 (malic enzyme 1; ME1) and malic enzyme 2 (malic enzyme 2; ME2) overexpression strains are constructed.

The plasmid pD4 (Mackenzie D A, Wongwathanarat P, Carter A T, et al. Isolation and use of a homologous histone H4 promoter and a ribosomal DNA region in a transformation vector for the oil-producing fungus Mortierella alpina[J]. Applied and environmental microbiology, 2000, 66(11): 4655-4661), pBIG2RHPH2 and Agrobacterium tumefaciens C58C1 (Tsuji G, Fujii S, Fujihara N, et al. Agrobacterium tumefaciens-mediated transformation for random insertional mutagenesis in Colletotrichum lagenarium[J]. Journal of General Plant Pathology, 2003, 69(4): 230-239) used in the present invention are publicly available.

The M. alpina uracil auxotrophic strain provides a prerequisite for gene manipulation of this PUFAs strain. The method of the present invention is based on the existing uracil auxotrophic strain, through genetic engineering methods to obtain a final phenotype complementary strain to achieve the malic enzyme 1 and malic enzyme 2 overexpression strains. The complementary strain is important for the further study on the relationship between malic enzyme and fatty acid synthesis in M. alpina, and can be used as candidate strains to produce high level of fatty acids.

The culture collection information of the present invention are as follows:

The A. tumefaciens C58C1 pBIG2-ura5s-malE1 generated in this invention has been maintained in China General Microbiological Culture Collection Center (CGMCC) since Sep. 24, 2013. The address of CGMCC is the Institute of Microbiology, Chinese Academy of Sciences, No. 1, Beichen West Road, Chaoyang District, Beijing, China, Zip code 100101. The accession number is CGMCC No. 8250.

The A. tumefaciens C58C1 pBIG2-ura5s-malE2 generated in this invention has been maintained in China General Microbiological Culture Collection Center (CGMCC) since Sep. 24, 2013. The address of CGMCC is the Institute of Microbiology, Chinese Academy of Sciences, No. 1, Beichen West Road, Chaoyang District, Beijing, China, Zip code 100101. The accession number is CGMCC No. 8261.

The A. tumefaciens C58C1 pBIG2-ura5s-ITs generated in this invention has been maintained in China General Microbiological Culture Collection Center (CGMCC) since Sep. 24, 2013. The address of CGMCC is the Institute of Microbiology, Chinese Academy of Sciences, No. 1, Beichen West Road, Chaoyang District, Beijing, China, Zip code 100101. The accession number is CGMCC No. 8249.

### BRIEF DESCRIPTION OF DRAWINGS

FIG. 1 is the schematic diagram of construction of the plasmid pBIG2-ura5s-malE1 that used for transformation of M. alpina.

FIG. 2 is the schematic diagram of construction of the plasmid pBIG2-ura5s-malE2 that used for transformation of M. alpina.

FIG. 3 is the schematic diagram of agarose gel electrophoresis of identification of the recombinant strains.

FIG. 4 is the schematic diagram of agarose gel electrophoresis of identification of the recombinant strains.

FIG. 5 is the diagram of the transcription level, translation level, enzymatic level and fatty acids analysis of ME1 strains overexpressing malE1.

FIG. 6 is the diagram of the transcription level, translation level, enzymatic level and fatty acids analysis of ME2 strains overexpressing malE2.

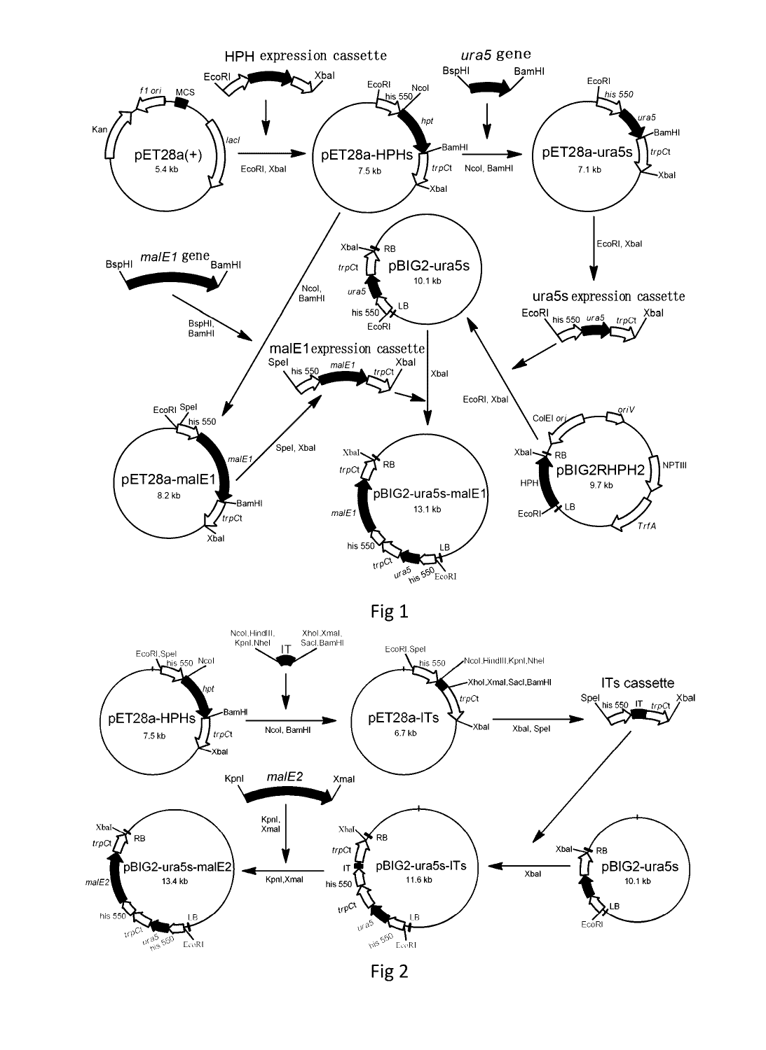

### EMBODIMENTS

The following Embodiments further illustrate the present invention. The experimental methods without indicating specific conditions in the followings examples will be performed generally in accordance with the manual of molecular cloning experiments.

### Example 1

### The Bioinformatics Analysis of M. alpina ATCC 32222 Genome

The protein coding sequence, which was predicted based on the M. alpina ATCC 32222 genome (DDBJ/EMBL/GenBank accession ADAG00000000, first version ADAG01000000), was compared to the database NR (www.ncbi.nlm.nih.gov), KOGs and COGs, KEGG, Swiss-Prot, UniRef100, and BRENDA using BLAST (E-value 1E-5). Search InterProScan against protein domain databases with default parameter settings. The 654 bp ura5 gene coding sequence was predicted. The 1752 bp malE1 gene coding sequence was predicted. The 1857 bp malE2 gene coding sequence was predicted.

### Example 2

### The Isolation of M. alpina Total RNA

(1) The frozen mycelia was grinded in a sterilized, enzyme free mortar;

(2) 1 mL of TRIzol (Invitrogen, Carlsbad, Calif., USA) was added and grinded, then placed at room temperature until the mixture was dissolved;

(3) 1 mL of the liquid in step (2) had been transferred to a new enzyme-free tube and 200 μL chloroform was added;

(4) The supernatant was transferred to a new enzyme free tube after centrifuged for 15 min at 12000 rpm at 4° C.;

(5) Equal volume of isopropanol was added and placed for 15 min, then centrifuged at 12000 rpm for 15 min at 4° C.;

(6) isopropanol was discarded;

(7) The pellet was washed once with 70% ethanol, then centrifuged at 12000 rpm for 15 min at 4° C.;

(8) The pellet was dissolved with RNase-free water, and stored at −80° C.;

(9) Determination of the concentration: Two microliters of RNA solution was used to determine the concentration of total RNA with Nanodrop2000;

(10) Agarose gel: One microliters of RNA solution was used to examine the completeness of total RNA using 1.2% agarose gel.

### Example 3

### Acquiring the Ura5 Gene, malE1 Gene, malE2 Gene and IT DNA Fragment

(1) The total RNA (0.5 to 1 μL) was reverse transcribed to cDNA using and following the instructions of PrimeScript RT reagent kit (TaKaRa, Otsu, Shiga, Japan);

(2) Based on the bioinformatics analysis results, the ura5 gene, malE1 gene, malE2 gene and IT DNA fragment specific primers were designed (the restrict sites were indicated by the underline):

URA5F:
(SEQ ID NO. 5)
ACATCATGACCATCAAGGAATACCAGCGCG
URA5R:
(SEQ ID NO. 6)
TCGGGATCCCTAAACACCGTACTTCTCC
malE1F:
(SEQ ID NO. 7)
CATGCGTCATGACTGTCAGCGAAAACACC
malE1R:
(SEQ ID NO. 8)
TACGCGGATCCTTAGAGGTGAGGGGCAAAGG
malE2F:
(SEQ ID NO. 9)
ATCGGGGTACCATGTTGAGGAATCCTGCTCTCA
malE2R:
(SEQ ID NO. 10)
TAATTCCCCCGGGTCAGGGGTGCGATTCCAG
ITF:
(SEQ ID NO. 11)
GCATGCCATGGAGAAGCTTGGTACCGCTAGCTCCCAAGCGAATTTGTCAT
CTCG
ITR:
(SEQ ID NO. 12)
CGCGGATCCGAGCTCCCCGGGGGACTCGAGAGCATACGGAAGTCCATCAG
TTACG

(3) The genes were amplified by PCR with cDNA or genome DNA as template;

(4) The DNA fragments were ligated into pEGM-T easy vector (Promega, Mandison, Wis., USA), and the sequence were analyzed with sequence by ABI PRISM 3730. Then the T-vectors were maintained in Escherichia coli TOP10 at −80° C.

### Example 4

### Construction of the Selected Marker Plasmid pBIG2-ura5s

Primers were designed based on the sequence
of plasmid pD4:
HPHF:
(SEQ ID NO. 17)
GAGACGAATTCGCCCGTACGGCCGACTAGTTTTAGTTGATGTGAG
HPHR:
(SEQ ID NO. 18)
GTTCCTCGTCTAGACCTCTAAACAAGTGTACCTGTGCATTCTGGG

The HPH expression cassette was PCR amplified.

The HPH expression cassette and plasmid pET28a were digested with EcoRI and XbaI, purified and ligated with T4 DNA ligase. The 10 μL ligation mixture containing: 2 μL of HPH DNA fragment, 1 μL of pET28a, 1 μL of 10× ligase buffer, 1 μL of T4 ligase and 5 μL of sterile water, incubated at 4° C. overnight.

The ligation product was directly transformed into E. coli TOP10 competent cell. The electro transformation steps are detailed below:

(1) 100 μL competent cells were taken out under sterile conditions, and 1 to 2 μL ligation product was added and mixed;

(2) The mixture of step (1) was transferred into a cuvette, avoiding making air bubbles;

(3) The cuvette was transferred into the Bio-Rad electroporation device and then the appropriate program and click pulse were selected;

(4) The pulsed competent cell was transferred into 900 μL SOC medium and incubated at 37° C. for 1 h;

(5) 200 μL of the culture was transferred onto YEP plate (containing 100 μg/mL kanamycin) and spread with a sterile stick;

The positive transformant was selected, and then the plasmid was extracted. The sequence was analyzed by ABI PRISM 3730. The resulted plasmid named pET28a-ura5s.

The ura5 expression cassette was PCR amplified with pET28a-ura5s as template using primer pair HPHF/HPHR.

The ura5 expression cassette was digested with SpeI and XbaI and the plasmid pBIG2RHPH2 was digested with XbaI, then purified and ligated with T4 ligase. The ligation product was directly transformed into E. coli TOP10 competent cell and the positive transformants were selected and sequence analyzed by ABI PRISM 3730. The resulting plasmid named pBIG2-ura5s.

The SOC recover medium was composed of 20 g/L Tryptone, 5 g/L yeast extract, 0.5 g/L NaCl, 2.5 mM KCl, 10 mM MgCl₂ and 2.2 mM glucose. The YEP solid medium was composed of 10 g/L Tryptone, 10 g/L yeast extract, 5 g/L NaCl and 20 g/L agar.

### Example 5

### Construction of the ME1 Expression Plasmid pBIG2-ura5s-malE1

The malE1 gene was digested with BspHI and BamHI, and the plasmid pET28a-HPHs were digested with NcoI and BamHI. The mixture was purified and ligated with T4 ligase. The ligation product was directly transformed into E. coli TOP10 competent cell and sequence analyzed by ABI PRISM 3730. The resulting plasmid named pET28a-malE1.

The malE1 expression cassette was PCR amplified with pET28a-malE1 as template using primer pair malE1F/malE1R.

The malE1 expression cassette and plasmid pBIG2-ura5s were digested with SpeI/XbaI and XbaI. The mixture was purified and ligated with T4 ligase. The ligation product was directly transformed into E. coli TOP10 competent cell and sequence analyzed by ABI PRISM 3730. The resulting plasmid named pBIG2-ura5s-malE1.

### Example 6

### Construction of the M. alpina Gene Manipulation Universal Vector pBIG2-ura5s-ITs and ME2 Expression Vector pBIG2-ura5s-malE2

The IT DNA fragment was PCR amplified with M. alpina genome as template using primer pair ITF/ITR.

The IT DNA fragment and plasmid pET28a-HPHs were digested with NcoI and BamHI. The mixture was purified and ligated with T4 ligase. The ligation product was directly transformed into E. coli TOP10 competent cell and sequence analyzed by ABI PRISM 3730. The resulting plasmid named pET28a-ITs.

The ITs cassette was obtained by SpeI and XbaI digestion of plasmid pET28a-ITs. The mixture was purified and ligated with T4 ligase. The ligation product was directly transformed into E. coli TOP10 competent cell and sequence analyzed by ABI PRISM 3730. The resulting plasmid was the M. alpina gene manipulation universal vector pBIG2-ura5s-ITs.

The malE2 DNA fragment and plasmid pBIG2-ura5s-ITs were digested with KpnI and XmaI. The mixture was purified and ligated with T4 ligase. The ligation product was directly transformed into E. coli TOP10 competent cell and sequence analyzed by ABI PRISM 3730. The resulting plasmid named pBIG2-ura5s-malE2.

### Example 7

### The ATMT of M. alpina

The transformation was optimized and essentially according to the method referred to the open accessed articles, the detailed steps are as follows:

(1) The A. tumefaciens C58C1 (harboring plasmid) was taken out from −80° C. and separated by stripping on the YEP solid plate (containing 100 μg/mL rifampicin and 100 μg/mL kanamycin) to obtain single clone by cultured at 30° C. for 48 h;

(2) A single clone was transferred to 20 mL YEP medium (containing 100 μg/mL rifampicin and 100 μg/mL kanamycin) and cultured at 30° C. for 48 h with shaking at 200 rpm in the dark;

(3) A. tumefaciens were collected by centrifuging at 4000×g for 5 min. After removing the suspension, the pellet was suspended by 5 mL of IM medium, followed by a centrifugation at 4000×g for 5 min. After removing the suspension, 2 mL of IM medium was added to suspend the bacterium;

(4) The concentration of the bacterium suspension was adjusted to OD600=0.9, followed by a dark cultivation at 30° C. to OD600=1.5;

(5) The M. alpina (the Mortierella alpina ATCC 32222 uracil auxotroph disclosed in Chinese Patent Application No. 201310347934.8) spores were collected and the number was counted, then adjusted the spore concentration to 10⁷/100 μL;

(6) Equal volume of 100 μL of A. tumefaciens and spores were mixed and spread on the cellophane membrane that placed on the IM solid medium, incubated at 23° C. for 48 to 96 h in a dark incubator;

(7) The cellophane membrane was transferred onto GY plate containing 100 μg/mL cefotaxime and 100 μg/mL spectinomycin, then incubated at 25° C. to 30° C. until spores appeared;

(8) The visible mycelium was immediately transferred onto the SC plate containing 100 μg/mL cefotaxime and 100 μg/mL spectinomycin.

The liquid MM medium was composed of 1.74 g/L K₂HPO₄, 1.37 g/L KH₂PO₄, 0.146 g/L NaCl, 0.49 g/L MgSO₄.7H₂O, 0.078 g/L CaCl₂, 0.0025 g/L FeSO₄.7H₂O, 0.53 g/L (NH₄)₂SO₄, 7.8 g/L MES, 1.8 g/L glucose and 0.5% glycerol. The IM medium was based on MM medium supplemented with 200 μM acetosyringone (AS). The SC medium was composed of 5 g/L Yest Nitrogen Base w/o Amino Acids and Ammonium Sulfate, 1.7 g/L (NH₄)₂SO₄, 20 g/L glucose, 20 mg/L Adenine, 30 mg/L Tyrosine, 1 mg/L Methionine, 2 mg/L Histidine, 4 mg/L Lysine, 4 mg/L Tryptophan, 5 mg/L Threonine, 6 mg/L Isoleucine, 6 mg/L Leucine, 6 mg/L Phenylalanine and 2 mg/L Arginine.

### Example 8

### The Screening and Identification of Recombinant Strains

(1) The mycelium was transferred to SC plate and incubated at 25° C. to 30° C. for 3 to 5 days to allow the mycelium visibly grow;

(2) The newly grown mycelium was transferred onto fresh SC plate containing 100 μg/mL cefotaxime and 100 μg/mL spectinomycin;

(3) The surface of the plate was flushed with 3 mL of physiological saline and the liquid was collected in a 1.5 mL tube, and then filtered with a 25 μm membrane;

(4) The liquid of 200 μL was spread onto the SC plate containing 100 μg/mL cefotaxime and 100 μg/mL spectinomycin until spores generated. This step was repeated for 3 times;

(5) The grown colonies of step (4) was transferred onto GY plates containing 1 mg/mL 5-FOA or without 5-FOA, respectively. Cultured at 25° C. for 2 to 4 days;

(6) The growth of M. alpina mycelium was observed on both kind of plate. The mycelium that could not grow on GY plates containing 1 mg/mL 5-FOA was transferred onto fresh GY plates;

(7) The genome of M. alpina strains was extracted. Two pairs of the promoter and terminator specific primers were designed to PCR identification of the positive transformants:

| (SEQ ID NO. 25) | |
|---|---|
| HisproF1: | CACACACAAACCTCTCTCCCACT |
| (SEQ ID NO. 26) | |
| TrpCR1: | CAAATGAACGTATCTTATCGAGATCC |
| (SEQ ID NO. 27) | |
| HisproF2: | GTGTTCACTCGCATCCCGC |
| (SEQ ID NO. 28) | |
| TrpCR2: | AGGCACTCTTTGCTGCTTGG |

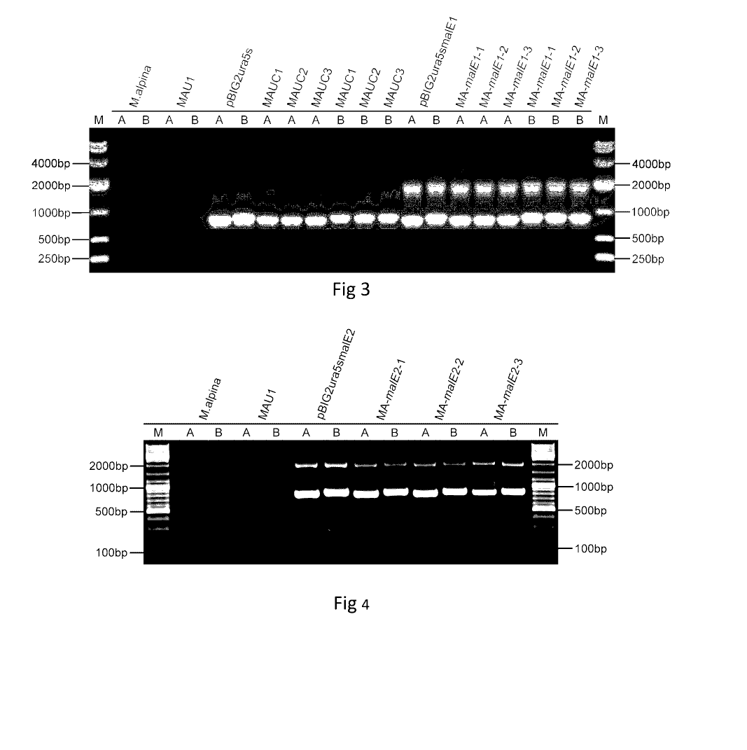

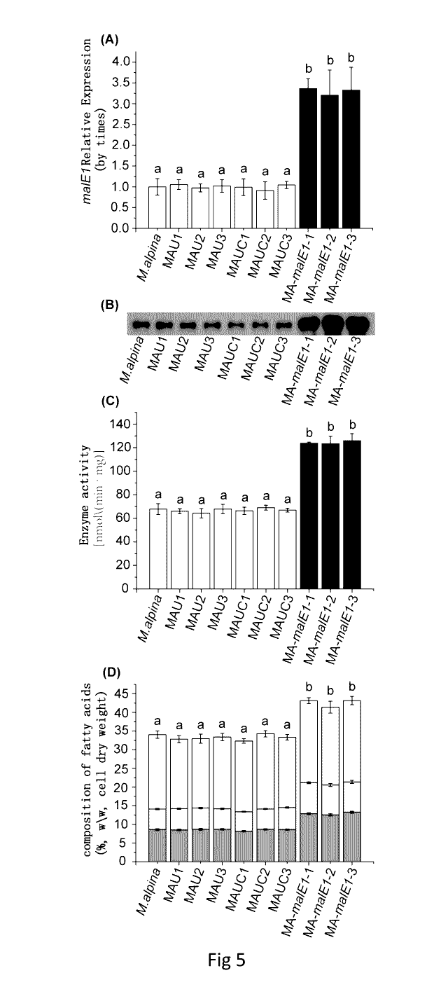

After the PCR reaction, strains were identified as recombinant strains (FIGS. 3 and 4). M represents the DNA marker; A represents the PCR product of primer pair HisproF1 and TrpCR1; B represents the PCR product of primer pair HisproF2 and TrpCR2. M. alpina represents the wild type control, MAU1 represents the blank control, and there are no PCR product detected. As shown in FIG. 3, pBIG2-ura5s and pBIG2-ura5s-malE1 represents the positive control that is the PCR products with plasmid as template. MAUC1, MAUC and MAUC3 are pBIG2-ura5s transformed recombinant strains that could be amplified to produce 818 bp and 861 bp band with primer pair A/B. This result is consistent with that of the pBIG2-ura5s positive control. MA-malE1-1, MA-malE1-2 and MA-malE1-3 are pBIG2-ura5s-malE1 transformed recombinant strains that could be amplified to produce 818 bp/1916 bp and 861 bp/959 bp band with primer pair A/B. This result is consistent with that of the pBIG2-ura5s-malE1 positive control. As shown in FIG. 4, pBIG2-ura5s-malE2 represents the positive control that is the PCR products with plasmid as template. MA-malE2-1, MA-malE2-2, MA-malE2-3 are pBIG2-ura5s-malE2 transformed recombinant strains that could be amplified to produce 818 bp/2021 bp and 861 bp/2064 bp band with primer pair A/B. This result is consistent with that of the pBIG2-ura5s-malE2 positive control.

(8) The recombinant strains were maintained on the GY medium.

### Example 9

### RT-qPCR Analysis of the Positive Transformants of malE1 Gene and malE2 Gene

Primers were designed according to the DNA sequence of malE1 gene, malE2 gene and 18SrDNA:

| (SEQ ID NO. 29) | |
|---|---|
| malE1RTF: | GGCTGTTGCCGAAGGGACT |
| (SEQ ID NO. 30) | |
| malE1RTR: | GGCAAAGGTGGTGCTGATTTC |
| (SEQ ID NO. 31) | |
| malE2RTF: | CCTTGCAGGACCGTAACGAGA |
| (SEQ ID NO. 32) | |
| malE2RTR: | CCTGGAGCGACGATAAATGGA |
| (SEQ ID NO. 33) | |
| 18SRTF: | CGTACTACCGATTGAATGGCTTAG |
| (SEQ ID NO. 34) | |
| 18SRTR: | C CGTACTACCGATTGAATGGCTTAG |

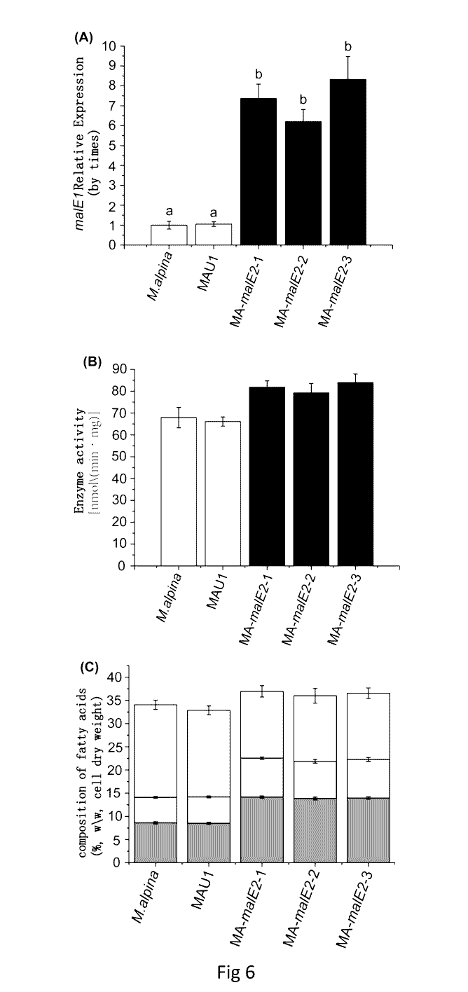

The cDNA of transformants was obtained following the method described in Example 2 and Example 3. RT-qPCR was performed on the ABI-Prism 7900 sequence detection system (Applied Biosystems, CA) with the Power SYBR Green PCR Master Mix (Applied Biosystems, CA). Reaction mixtures composed of 10 μL of SYBR Green PCR Master Mix, 0.5 μL of each primer, 8 μL of distilled water, and 1 μL of DNA template or distilled water as negative control were prepared. The PCR cycling conditions were 50° C. for 2 min, 95° C. for 10 min, followed by 40 cycles of amplification at 95° C. for 15 s and 60° C. for 30 s. The expression of the internal control gene (18S rRNA) was used as the normalization standard for gene expression. All of the samples were measured in triplicate. The result is illustrated in FIG. 5A. M. alpina represents the wild type control; MAU1, MAU2 and MAU3 represent the recipient control; MAUC1, MAUC2 and MAUC3 are pBIG2-ura5s recombinant strain, of which the expression of malE1 was not affected by the ura5 selective marker. MA-malE1-1, MA-malE1-2 and MA-malE1-3 are pBIG2-ura5s-malE1 recombinant strains of which the expression of malE1 was significantly increased compared to the control. The result is illustrated in FIG. 6A. M. alpina represents the wild type control; MAU1 represents the recipient control; MA-malE2-1, MA-malE2-2 and MA-malE2-3 are pBIG2-ura5s-malE2 recombinant strains of which the expression of malE2 was significantly increased compared to the control.

### Example 10

### Western Blot of ME1 Protein in Recombination Strain

(1) Cells were grinded with liquid nitrogen and total protein was extracted;

(2) The concentration of the proteins extract was measured. Then the proteins were separated via SDS-PAGE electrophoresis in Bio-Rad electrophoresis apparatus with 10 μg loading quantity in each lane;

(3) The separated proteins were transferred from the SDS-PAGE gel into PVDF membrane within the electrophoresis apparatus at 50 V for 3 h;

(4) The PVDF membrane was blocked in 5% skim milk in horizontal shaker at room temperature for 30 to 40 min;

(5) The PVDF membrane was continued to soak in TBST buffer in the shaker at room temperature for 10 min. This step was repeated for three times;

(6) The primary antibody (designed based on the gene sequence of ME1 and synthesized by Bio Basic inc.) was added into the TBST buffer at the ratio 1/3000 and the PVDF membrane was incubated on the horizontal shaker for 1 h;

(7) The PVDF membrane soaked into TBST buffer was incubated on the horizontal shaker at room temperature for 10 min. This step was repeated for three times;

(8) The secondary antibody was added into 5% skim milk at the ratio 1/3000 and continued to incubate the PVDF membrane for 1 h;

(9) The PVDF membrane soaked into TBST buffer for was incubated on the horizontal shaker at room temperature for 10 min. This step was repeated for three times;

(10) The protein in PVDF membrane was visualized through enhanced chemiluminescence (ECL) method. Use a film to expose the PVDF membrane in dark space.

The results are shown in FIG. 5B. M. alpina is the wild type. MAU1, MAU2 and MAU3 are recipient control. MAUC1, MAUC2 and MAUC3 are recombinant strains for plasmid pBIG2-ura5s. MA-malE1-1, MA-malE1-2 and MA-malE1-3 are recombinant strains for plasmid pBIG2-ura5s-malE1.

The result showed that the ME1 protein level in MA-malE1-1, MA-malE1-2 and MA-malE1-3 strains are much higher than the recipient control and the pBIG2-ura5s recombinant strains.

### Example 11

### Enzyme Activity Measurement of ME in the Recombinant Strains

(1) Cells were grinded with liquid nitrogen and total protein was extracted;

(2) The measurement system in UV spectrophotometer was set up. The buffer was composed of 80 mM KH₂PO₄/KOH pH 7.5, 0.6 mM NADP⁺, 3 mM MgCl₂ and rough protein samples (about 30 g protein);

(3) The system was maintained at 30° C. for 2 min. When the status was table, the malic enzyme (pH 6.8) was added into the system at final concentration 25 mM;

(4) The data was collected in 340 nm for 3 min. The enzyme activity was calculated based on the change of the absorb value per unit time.

The results are shown in FIG. 5C. M. alpina is the wide type. MAU1, MAU2 and MAU3 are recipient control. MAUC1, MAUC2 and MAUC3 are recombinant strains for plasmid pBIG2-ura5s. MA-malE1-1, MA-malE1-2 and MA-malE1-3 are recombinant strains for plasmid pBIG2-ura5s-malE1. In FIG. 6B, M. alpina is the wide type. MAU1 is recipient control. MA-malE2-1, MA-malE2-2 and MA-malE2-3 are recombinant strains for plasmid pBIG2-ura5s-malE2. As we can see from the FIGS. 5C and 6B, the activity of ME1 protein of those ME gene overexpression recombinant strains are significantly increased compared with control.

### Example 12

### Extraction and Analysis of the Fatty Acids of M. alpina

(1) The M. alpina strains were cultured in ferment medium at 25° C., 200 rpm for 144 h, with wild-type strain as control;

(2) Mycelia were collected and freeze-dried;

(3) One hundred milligram freeze-dried mycelia was mixed with 2 mL 4 mol/L HCl;

(4) The mixture was incubated at 80° C. for 0.5 h and −80° C. for 15 min. Repeated once. Then the mixture was incubated at 80° C. water bath for 0.5 h;

(5) The mixture was cooled down to room temperature, and then 1 mL methanol was added and mixed;

(6) 1 mL chloroform was added and shaken for 10 min, followed by centrifuge at 6000×g for 3 min. Chloroform was collected;

(7) Step (6) was repeated for two times;

(8) Chloroform (3 mL) was combined, 1 mL saturated NaCl solution was added, mixed and centrifuged at 3000×g for 3 min. Chloroform was collected into a new tube. 1 mL chloroform was added into the residual liquid, followed by centrifugation at 3000×g for 3 min. All the chloroform (4 mL) were combined;

(9) After drying by nitrogen blow, 1 mL ethyl ether was added. The solution was transferred to a clean and weighed tube, followed by drying by nitrogen blow and weighed to obtain total fatty acid weight.

(10) The fatty acids were analyzed by GC. The results are shown in FIGS. 5D and 6C. Gray bars represent AA; light gray bars represent ω6-PUFAs, and white bars represent other fatty acids. M. alpina is the wide type control. MAU1, MAU2 and MAU3 are recipient control. MAUC1, MAUC2 and MAUC3 are recombinant strains for plasmid pBIG2-ura5s. MA-malE1-1, MA-malE1-2 and MA-malE1-3 are recombinant strains for plasmid pBIG2-ura5s-malE1. MA-malE2-1, MA-malE2-2 and MA-malE2-3 are recombinant strains for plasmid pBIG2-ura5s-malE2. As shown in FIG. 5D, the fatty acid content of recombinant strains MA-malE1-1, MA-malE1-2 and MA-malE1-3 have been improved by 30% compared to the control, and the content of AA was also increased. As shown in FIG. 6C, the total fatty acid content of recombinant strains MA-malE2-1, MA-malE2-2 and MA-malE2-3 were not improved, but the content of AA significantly increased.

The ferment medium was composed of 50 g/L glucose, 2.0 g/L L-Ammonium tartrate, 7.0 g/L KH₂PO₄, 2.0 g/L Na₂HPO₄, 1.5 g/L MgSO₄.7H₂O, 1.5 g/L Yeast extract, 0.1 g/L CaCl₂.2H₂O, 8 mg/L FeCl₃.6H₂O, 1 mg/L ZnSO₄.7H₂O, 0.1 mg/L CuSO₄.5H₂O, 0.1 mg/L Co(NO₃)₂.6H₂O, and 0.1 mg/L MnSO₄.5H₂O.

While the present invention has been disclosed as the preferred embodiment above, they are not intended to limit the present invention. Those skilled in the art, without departing from the spirit and scope of the present invention, can make various changes and modifications. Therefore, the protection scope of the present invention should be defined only by the claims.

What is claimed is:

1. A homologous recombinant Mortierella alpina strain overexpressing a malic enzyme gene, characterized in that the strain is constructed by transforming M. alpina uracil auxotroph strain using Agrobacterium tumefaciens containing malic enzyme gene, and the said Agrobacterium tumefaciens containing malic enzyme gene harbors the plasmid pBIG2-ura5s-malE1 or pBIG2-ura5s-malE2;
The said plasmid pBIG2-ura5s-malE1 is constructed with the following steps: The HPH expressing cassette is PCR amplified from pD4 plasmid and digested with EcoRI and XbaI, followed by insertion into the multiple cloning site (MCS) of pET28a (+) digested with EcoRI and XbaI, to form plasmid pET28a-HPHs; subsequently, the ura5 gene is digested with BspHI and BamHI, and the digested ura5 gene is inserted into pET28a-HPHs digested with NcoI and BamHI, to replace hpt gene to form pET28a-ura5s; the ura5s expressing cassette is obtained by digesting plasmid pET28a-ura5s with EcoRI and XbaI; replace HPH expressing cassette in pBIG2RHPH2 with the resultant ura5s expressing cassette to form transformant plasmid pBIG2-ura5s; the ma/E1 gene segment is digested with BspHI and BamHI, and pET28a-HPHs is digested with NcoI and BamHI, respectively, and ma/E1 gene segment is inserted into NcoI and BamHI site of plasmid pET28a-HPHs by ligation reaction to form plasmid pET28a-malE1; the ma/E1 expressing cassette is obtained by double digesting plasmid pET28a-malE1 with SpeI and XbaI; the malE1 expressing cassette is inserted into pBIG2-ura5s digested with XbaI to form plasmid pBIG2-ura5s-malE1;
The said plasmid pBIG2-ura5s-malE2 is constructed with the following steps: the IT noncoding intron DNA segment is achieved by PCR method from M. alpina genome, IT gene segment and plasmid pET28a-HPHs is digested with NcoI and BamHI respectively, then replace hpt gene in the pET28a-HPHS with IT segment by ligation reaction to obtain plasmid pET28a-Its, then pET28a-Its is double digested with SpeI and XbaI, to obtain ITs expression unit; the resultant ITs expression unit is inserted into pBIG2-ura5s digested by XbaI, to form the M. alpina gene manipulation common vector pBIG2-ura5s-ITs; the malE2 gene is double digested with KpnI and XmaI, then conduct ligation with ligase, to form malE2 expression plasmid pBIG2-ura5s-malE2.

2. The homologous recombinant M. alpina strain according to claim 1, characterized in that the strain is constructed by the transformation of A. tumefaciens using recombinant plasmid pBIG2-ura5s-malE1 or pBIG2-ura5s-malE2, followed by the transformation of M. alpina uracil auxotroph strain using transformed A. tumefaciens harboring the plasmid pBIG2-ura5s-malE1 or pBIG2-ura5s-malE2, the said M. alpina uracil auxotroph strain is M. alpina ATCC 32222 with the 18 bp (213 bp-230 bp) deletion in the ura5 gene.

3. A method of constructing the homologous recombination M. alpina strain, characterized in that the said method comprises the following steps:
a) extracting the RNA of M. alpina ATCC 32222, to obtain cDNA through the reverse transcription, and then obtain ura5 gene, IT intron DNA segment and malic enzyme genes malE1 and malE2 respectively by PCR, using cDNA as the template;
b) constructing the recombinant plasmids pBIG2-ura5s-malE1 and pBIG2-ura5s-malE2 seperately;
c) transforming A. tumefaciens using the constructed plasmid pBIG2-ura5s-malE1 or pBIG2-ura5s-malE2;
d) transforming the M. alpina uracil auxotrophic strain using the transformed A. tumefaciens harboring plasmid pBIG2-ura5s-malE1 or pBIG2-ura5s-malE2;
e) screening and identifying the transformant strains to obtain the homologous recombinant Mortierella alpina strain overexpressing malic enzyme 1 or malic enzyme 2 genes.

4. The method according to claim 3, characterized in that the A. tumefaciens used in step c) is Agrobacterium tumefaciens C58C1.

5. The method according to claim 3, characterized in that the M. alpina uracil auxotroph strain used in step d) is M. alpina ATCC 32222 with the 18 bp (213 bp-230 bp) deletion in the ura5 gene.

6. The method according to claim 3, characterized in that sequence of the primers, used for amplifying the ura5 gene IT in the step a), as well as that of the malic enzyme gene 1 malE1 and that of the malic enzyme gene 2 malE2, are as described below:
URA5F:
ACATCATGACCATCAAGGAATACCAGCGCG
URA5R:
TCGGGATCCCTAAACACCGTACTTCTCC
malE1F:
CATGCGTCATGACTGTCAGCGAAAACACC
malE1R:
TACGCGGATCCTTAGAGGTGAGGGGCAAAGG
malE2F:
ATCGGGGTACCATGTTGAGGAATCCTGCTCTCA
malE2R:
TAATTCCCCCGGGTCAGGGGTGCGATTCCAG
ITF:
GCATGCCATGGAGAAGCTTGGTACCGCTAGCTCCCAAGCGAATTTGTCAT
CTCG
ITR:
CGCGGATCCGAGCTCCCCGGGGGACTCGAGAGCATACGGAAGTCCATCAG
TTACG.
